(19) United States
(12) Patent Application Publication
(10) Pub. No.: US 2009/0318681 A1
(43) Pub. Date: Dec. 24, 2009

(54) METAL COMPLEX, AND USE THEREOF

(75) Inventors: Tadafumi Matsunaga, Ibaraki (JP); Nobuyoshi Koshino, Ibaraki (JP); Hideyuki Higashimura, Ibaraki (JP)

Correspondence Address: SUGHRUE MION, PLLC, 2100 PENNSYLVANIA AVENUE, N.W., SUITE 800, WASHINGTON, DC, 20037, US

(73) Assignee: Sumitomo Chemical Company Limited, Tokyo (JP)

(21) Appl. No.: 12/518,396

(22) PCT Filed: Dec. 11, 2007
(86) PCT No.: PCT/JP2007/074191

§ 371 Date: Jun. 9, 2009

(30) Foreign Application Priority Data
Dec. 11, 2006 (JP) 2006-333067
Aug. 7, 2007 (JP) 2007-205957

(51) Int. Cl. C07D 257/10; C07D 487/18

(52) U.S. Cl. 540/465; 540/472

(57) ABSTRACT

Provided is a metal complex represented by the following formula (A-1) or (B-1) as a metal complex useful for a redox reaction catalyst or some other article that is excellent in heat resistance and acid resistance:

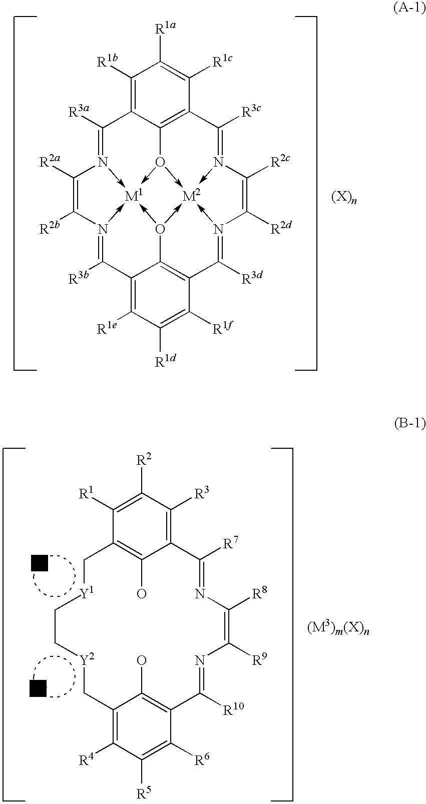

### TECHNICAL FIELD

The present invention relates to a metal complex, more specifically, a metal complex useful as a catalyst.

### BACKGROUND ART

Metal complexes each act a catalyst in a redox reaction involving electron transfer, such as oxygenation reaction, oxidative coupling reaction, dehydrogenation reaction, hydrogenation reaction, oxide decomposing reaction or electrode reaction, and are each used to produce an organic compound or a polymer compound. Furthermore, metal complexes are used in various applications such as an additive, a modifier, a cell, and a sensor material.

In particular, about redox reaction catalysts, it is known that Schiff base metal complexes have a highly active and highly selective catalyst potency. For example, in Org. Biomol. Chem., 2005, 3, 2126, an optically active Schiff base complex is used to oxidize the double bond of styrene to conduct an asymmetric reaction for yielding cyclopropane and the good asymmetric reaction advances. In Inorg. Chem., 2001, 40, 1329, a Schiff base metal complex is used to produce water by electrolytic reduction of oxygen. Angew. Chem. Int. Ed., 2003, 42, 6008 reports that an optically active Schiff base binuclear copper complex catalyst causes asymmetric oxygen oxidation of naphthol.

However, when this is used as a catalyst, each of the metal complexes disclosed in the individual documents above, may become instable and come to have a low activity under heating. Furthermore, it is feared that in the presence of a strong acid, the catalyst also becomes instable. Thus, an applicable scope of the catalyst is restricted. In such a manner, metal complexes known in the prior art may be decomposed depending on reaction conditions.

### DISCLOSURE OF THE INVENTION

The present invention provides a metal complex useful as a redox catalyst or some other applications, which is stable even at high temperature or in the presence of a strong acid, that is, is excellent in heat resistance and acid resistance.

The inventors have made eager investigations to solve the problems, so as to make the invention.

Accordingly, the invention provides metal complexes, a polymer and catalysts described in the following [1] to [1,3]:

[1] A metal complex represented by the following formula (A-1):

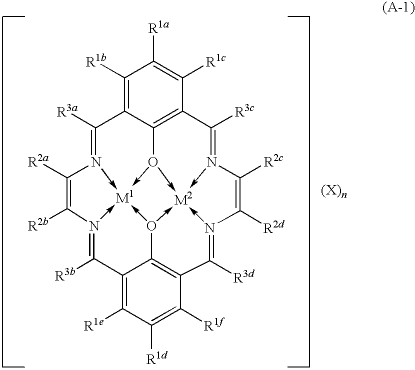

wherein one of M¹ and M² represents a transition metal atom belonging to Groups 6 to 9 of the long-period form periodic table, the other thereof represents a transition metal atom belonging to Groups 6 to 11 of the long-period form periodic table, and M¹ and M² may be the same as or different from each other; R^{1a} to R^{1f}, R^{2a} to R^{2d}, and R^{3a} to R^{3d} each independently represent a hydrogen atom or a substituent, and two substituents of each of pairs of R^{1a} and R^{1b}, R^{1a} and R^{1c}, R^{1d} and R^{1e}, R^{1d} and R^{1f}, R^{2a} and R^{2b}, R^{2c} and R^{2d}, R^{1b} and R^{3a}, R^{1c} and R^{3c}, R^{1e} and R^{3b}, and R^{1f} and R^{3d} may be linked to each other to form a ring; X is a counter ion which makes the metal complex electrically neutral, or a neutral molecule; n is the number of X(s) present in the complex and represents an integer of 0 to 4 provided that when n is an integer of 2 to 4, a plural of Xs may be the same as or different from each other; and the symbols “A” each represent a coordinate bond or ion bond to M¹ or M².

[2] The metal complex according to [1], wherein at least one of M¹ and M² is a transition metal ion belonging to the fourth period of the long-period form periodic table.

[3] A metal complex represented by the following formula (A-2):

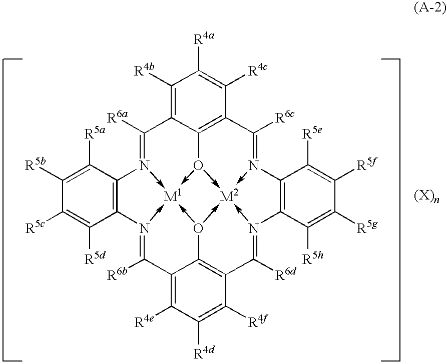

wherein one of M¹ and M² represents a transition metal atom belonging to Groups 6 to 9 of the long-period form periodic table, the other thereof represents a transition metal atom belonging to Groups 6 to 11 of the long-period form periodic table, and M¹ and M² may be the same as or different from each other; R^{4a} to R^{4f}, R^{5a} to R^{5h}, and R^{6a} to R^{6d} each independently represent a hydrogen atom or a substituent, and two substituents of each of pairs of R^{4a} and R^{4b}, R^{4a} and R^{4c}, R^{4d} and R^{4e}, R^{4d}, and R^{5a} and R^{5b}, R^{5b} and R^{5c}, R^{5c} and R^{5d}, R^{5e} and R^{5f}, R^{5f} and R^{5g}, R^{5g} and R^{5h}, R^{4b} and R^{6a}, R^{4e} and R^{6b}, R^{4c} and R^{6c}, and R^{4f} and R^{6d} may be linked to each other to form a ring; X is a counter ion which makes the metal complex electrically neutral, or a neutral molecule; n is the number of X(s) present in the complex and represents an integer of 0 to 4 provided that when n is an integer of 2 to 4, a plural of Xs may be the same as or different from each other; and the symbols “→” each represent a coordinate bond or ion bond to M¹ or M².

[4] The metal complex according to [3], wherein at least one of M¹ and M² is a transition metal ion belonging to the fourth period of the long-period form periodic table.

[5] A metal complex represented by the following formula (A-3):

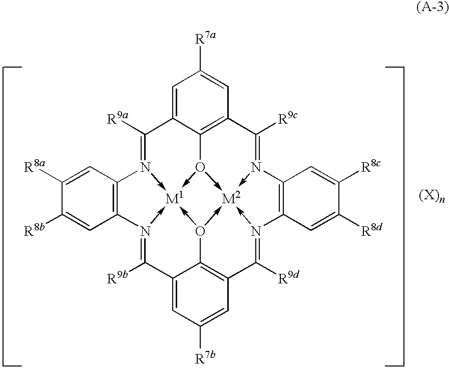

wherein one of M¹ and M² represents a transition metal atom belonging to Groups 6 to 9 of the long-period form periodic table, the other thereof represents a transition metal atom belonging to Groups 6 to 11 of the long-period form periodic table, and M¹ and M² may be the same as or different from each other; R^{7a}, R^{7b}, R^{8a} to R^{8d} and R^{9a} to R^{9d} each independently represent a hydrogen atom or a substituent, and two substituents of each of pairs of R^{8a} and R^{8b}, and R^{8c} and R^{8d} may be linked to each other to form a ring; X is a counter ion which makes the metal complex electrically neutral, or a neutral molecule; n is the number of X(s) present in the complex and represents an integer of 0 to 4 provided that when n is an integer of 2 to 4, a plural of Xs may be the same as or different from each other; and the symbols “→” each represent a coordinate bond or ion bond to M¹ or M².

[6] The metal complex according to [5], wherein at least one of M¹ and M² is a transition metal ion belonging to the fourth period of the long-period form periodic table.

[7] A metal complex represented by the following formula (B-1):

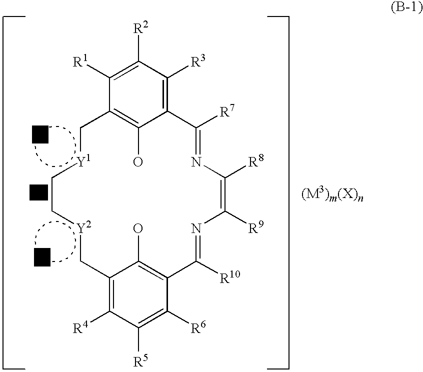

wherein R¹ to R¹⁰ each independently represent a hydrogen atom or a substituent; two substituents of each of pairs of R¹ and R², R² and R³, R⁴ and R⁵, R⁵ and R⁶, R³ and R⁷, R⁶ and R¹⁰, and R⁸ and R⁹ may be linked to each other to form a ring; Y¹ and Y² each independently represent

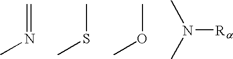

wherein R_{α} is a hydrogen atom or a hydrocarbon group having 1 to 4;
P¹ is a group of atoms necessary for being combined with Y¹ and carbon atoms adjacent thereto so as to form an aromatic heterocyclic ring, P² is a group of atoms necessary for being combined with Y² and carbon atoms adjacent thereto so as to form an aromatic heterocyclic ring, and P¹ and P² may be linked to each other to form an additional ring; M³ represents a transition metal atom or a typical metal atom; m represents 1 or 2 provided that when m is 2, two M³s may be the same as or different from each other; X is a counter ion which makes the metal complex electrically neutral, or a neutral molecule; and n is the number of X(s) present in the complex and represents an integer of 0 or more provided that when n is an integer of 2 or more, a plural of Xs may be the same as or different from each other.

[8] The metal complex according to [7], wherein M³ is a transition metal atom.

[9] A metal complex represented by the following formula (B-2):

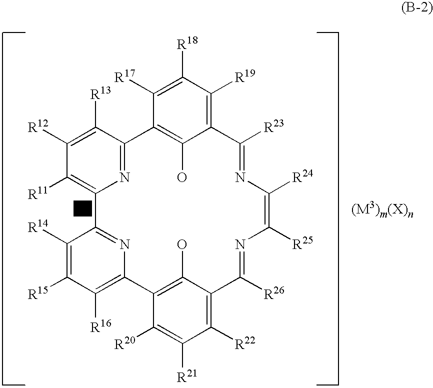

wherein R¹¹ to R²⁵ each independently represent a hydrogen atom or a substituent; two substituents of each of pairs of R¹¹ and R¹⁴, R¹¹ and R¹², R¹² and R¹³, R¹³ and R¹⁷, R¹⁴ and R¹⁵, R¹⁵ and R¹⁶, R¹⁶ and R²⁰, R¹⁷ and R¹⁸, R¹⁸ and R¹⁹, R²⁰ and R²¹, R²¹ and R²², and R²⁴ and R²⁵ may be linked to each other to form a ring; M³ represents a transition metal atom or a typical metal atom; m represents 1 or 2 provided that when m is 2, two M³s may be the same as or different from each other; X is a counter ion which makes the metal complex electrically neutral, or a neutral molecule; and n is the number of X(s) present in the complex and represents an integer of 0 or more provided that when n is an integer of 2 or more, a plural of Xs may be the same as or different from each other.

[10] The metal complex according to [9], wherein M³ is a transition metal atom.

[11] A polymer comprising a moiety obtained by removing, from a metal complex as recited in any one of [1] to [10], its hydrogen atom or its substituent.

[12] A catalyst comprising a metal complex as recited in any one of [1] to [10].

[13] A catalyst comprising a polymer as recited in [11].

The metal complexes of the invention are excellent in heat resistance and acid resistance. Accordingly, the metal complexes are restrained from deterioration of their catalyst activity even in the presence of a strong acid or at high temperature. For this reason, the metal complexes can become catalysts which has a wide scope for use; thus, the complexes are industrially useful.

### BRIEF DESCRIPTION OF THE DRAWINGS

FIG. 1 is an IR absorption spectrum of a metal complex (A).

FIG. 2 is an IR absorption spectrum of a metal complex (B).

FIG. 3 is an IR absorption spectrum of a metal complex (C).

FIG. 4 is an IR absorption spectrum of a metal complex (D).

FIG. 5 is an IR absorption spectrum of a metal complex (E).

### BEST MODE FOR CARRYING OUT THE INVENTION

The metal complex represented by the formula (A-1), which is a first embodiment of the invention, is described. The metal complex is a product wherein two transition metal atoms (M¹ and M²) form a complex by a ligand having four nitrogen atoms and two oxygen atoms, and n electrons present in the ring having the four nitrogen atoms are non-localized. The bond through which any one of the oxygen atoms and any one of the metal atoms are linked to each other is a coordinate bond or ion bond. Bridging coordination may be attained between the two transition metals. The word “transition metal” has the same meanings as the substance described as “transition element” in p. 1283 of “Kagaku Dai-Jiten (Chemical Great Dictionary)” (edited by Michinori Ohgi et al., and published by Tokyo Kagaku Dozin Co., Ltd. on Jul. 1, 2005), and means any element having an incomplete d or f sub-shell. Any transition metal atom in the invention may be in a non-charged state or in a charged ion state.

About M¹ and M², one thereof is a transition metal atom belonging to Groups 6 to 9 of the long-period form periodic table (IUPAC 2001), and the other thereof is a transition metal atom belonging to Groups 6 to 11 of the long-period form periodic table (IUPAC 2001).

Specific examples of the former transition metal atom include chromium, manganese, iron, cobalt, molybdenum, technetium, ruthenium, rhodium, tungsten, rhenium, osmium, and iridium. The metal atom is preferably chromium, manganese, iron, cobalt, molybdenum, ruthenium or rhodium, more preferably chromium, manganese, iron or cobalt, even more preferably manganese, iron or cobalt. The former transition metal atom is preferably a transition metal ion belonging to the forth period of the above-mentioned periodic table.

Specific examples of the latter transition metal atom include nickel, copper, palladium, silver, platinum and gold besides the examples of the former transition metal atom. The metal atom is preferably chromium, manganese, iron, cobalt, nickel, copper, molybdenum, ruthenium, rhodium, palladium or silver, more preferably chromium, manganese, iron, cobalt, nickel or copper, even more preferably manganese, iron, cobalt, nickel or copper. The latter transition metal atom is also preferably a transition metal ion belonging to the forth period of the above-mentioned periodic table.

The following describes the ligand of the metal complex represented by the formula (A-1). The ligand has four nitrogen atoms and two oxygen atoms as coordinating atoms, as described above. About the ring having the four nitrogen atoms, π electrons on the ring are non-localized, that is, the ring being π conjugated. This ring may have a substituent, and R^{1a} to R^{1f}, R^{2a} to R^{2f}, and R^{3a} to R^{3d} in the formula (A-1) each independently represent a hydrogen atom or a substituent.

Examples of the substituent include halogeno radicals such as fluoro, chloro, bromo and iodo radicals, a hydroxy group, a carboxyl group, a mercapto group, a sulfonic acid group, a nitro group, a phosphonic acid group, silyl groups each having an alkyl group having 1 to 3 carbon atoms, linear, branched and cyclic saturated hydrocarbon groups having about 1 to 50 carbon atoms as a whole, such as methyl group, ethyl group, propyl group, isopropyl group, cyclopropyl group, butyl group, isobutyl group, t-butyl group, pentyl group, cyclopentyl group, hexyl group, cyclohexyl group, norbornyl group, nonyl group, cyclononyl group, decyl group, 3,7-dimethyloctyl group, adamanthyl group, dodecyl group, cyclododecyl group, pentadecyl group, octadecyl group and docosyl group; linear, branched and cyclic alkoxy groups having about 1 to 50 carbon atoms as a whole, such as methoxy group, ethoxy group, propoxy group, butoxy group, pentyloxy group, cyclohexyloxy group, norbornyloxy group, decyloxy group and dodecyloxy group; and aromatic groups having about 3 to 60 carbon atoms as a whole, such as phenyl group, 4-methylphenyl group, 1-naphthyl group, 2-naphthyl group, pyridyl group, furyl group, oxazolyl group, imidazolyl group, pyrazolyl group, pyrazyl group, pyrimidyl group, pyridazyl group and benzoimidazolyl group.

R^{1a} to R^{1f}, R^{2a} to R^{2f}, and R^{3a} to R^{3d} are each preferably a halogeno radical such as a fluoro, chloro, bromo or iodo radical, a mercapto group, a hydroxy group, a carboxyl group, a hydrocarbon group having about 1 to 20 carbon atoms as a whole, such as a methyl group, ethyl group, propyl group, isopropyl group, butyl group, pentyl group, tert-butyl group, cyclohexyl group, norbornyl group or adamanthyl group, a linear or branched alkoxy group having about 1 to 10 carbon atoms as a whole, such as a methoxy group, ethoxy group, propoxy group, butoxy group or pentyloxy group, or an aromatic group having about 6 to 30 carbon atoms as a whole, such as a phenyl group, 1-naphthyl group, 2-naphthyl group or 9-anthryl group.

R^{1a} to R^{1f}, R^{2a} to R^{2f}, and R^{3a} to R^{3d} are each more preferably a chloro or bromo radical, or a hydroxyl group, carboxyl group, methyl group, ethyl group, tert-butyl group, cyclohexyl group, norbornyl group, adamanthyl group, methoxy group, ethoxy group or phenyl group.

Two substituents of each of pairs of R^{1a} and R^{1b}, R^{1a} and R^{1c}, R^{1d} and R^{1e}, R^{1d} and R^{1f}, R^{2a} and R^{2b}, R^{2c} and R^{2d}, R^{1b} and R^{3a}, R^{1c} and R^{3c}, R^{1e} and R^{3b}, R^{1f} and R^{3d}, R^{2a} and R^{3a}, and R^{2b} and R^{3b} may be linked to each other to form a ring

Examples of the ring include hydrocarbon rings such as cyclohexene ring, benzene ring, naphthalene ring, anthracene ring, tetracene ring, perylene ring, pentacene ring and acenaphthene ring; and aromatic heterocyclic rings such as pyran, furan ring, pyridine ring, pyrazine ring, pyrazolyl ring, imidazolyl ring, oxazole ring, isooxazole ring, thiazole ring, isothiazole ring and thiophene ring. The ring is preferably a benzene ring, naphthalene ring, pyridine ring or pyrazine ring, in particular preferably a benzene or naphthalene, most preferably a benzene ring.

The ring formed by linking the two substituents of any one of the pairs to each other may further have a substituent. Examples of the substituent can be the same substituents as exemplified above.

In the metal complex of the invention, it is preferred that one or more out of the two-substituent pairs exemplified above (each) form a ring. This case causes the heat resistance of the metal complex to be further improved.

Examples of the metal complex represented by the formula (A-1) include metal complexes having ligand skeleton structures (a-I) to (a-XI) illustrated below, Their transition metal atoms are not illustrated, and their electric charges are omitted. In the examples illustrated below, Me, Et and t-Bu represent methyl, ethyl and tert-butyl groups, respectively.

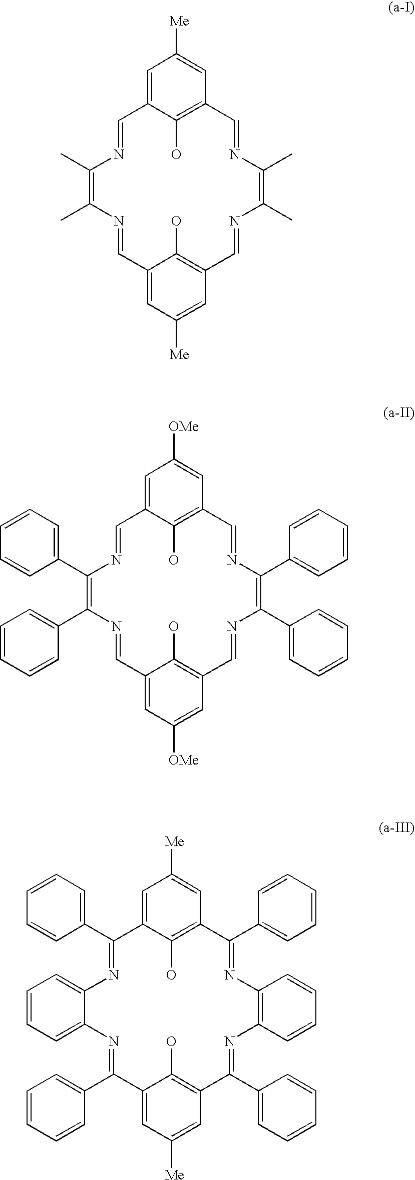

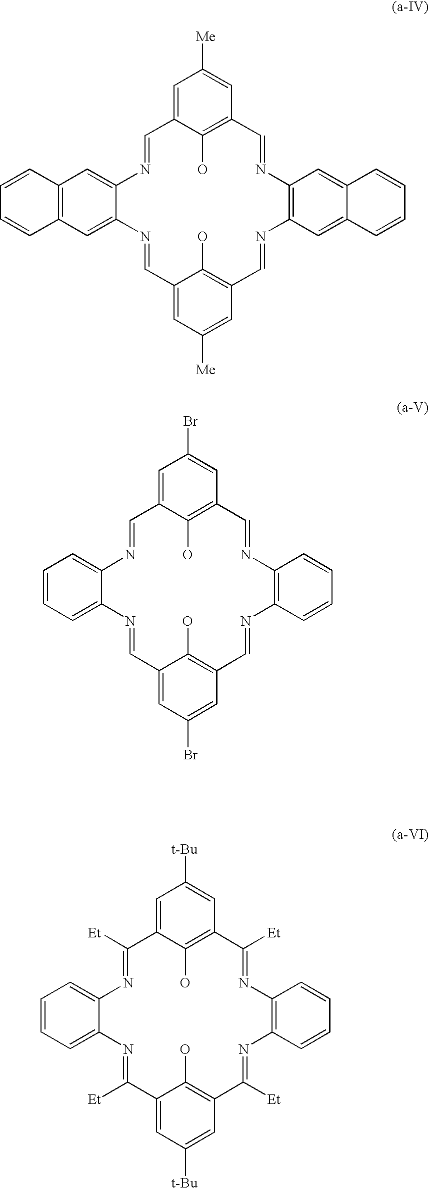

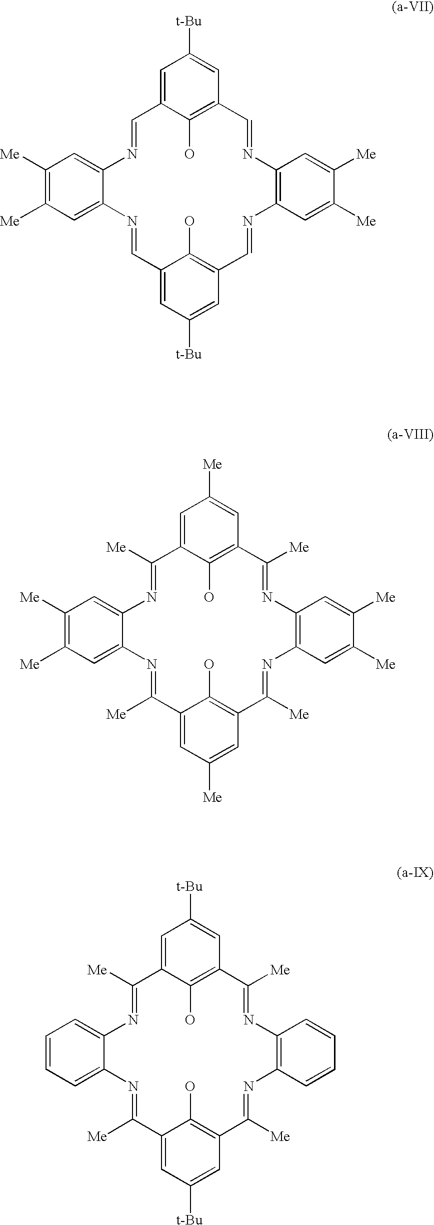

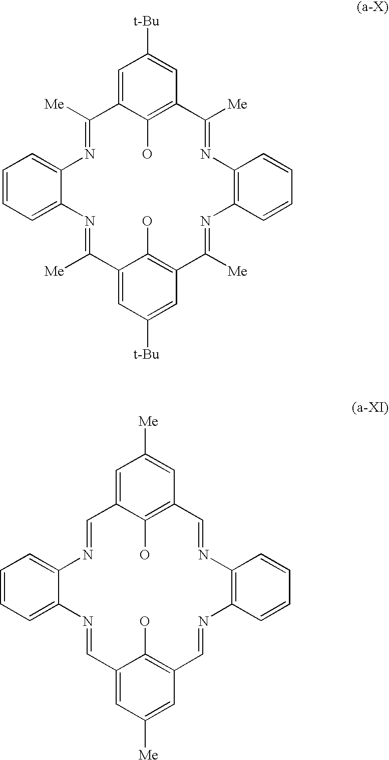

As the conjugation length of the n electrons non-localized in the metal complex represented by the formula (A-1) is longer, the heat resistance can be made better; thus, a case where the length is longer is preferred, Specifically, the metal complex is preferably a metal complex in which in one or more out of the above-mentioned two-substituent pairs, the ring(s) wherein the two substituents are linked to each other (each) has/have a ligand which is an aromatic homocyclic ring or aromatic heterocyclic ring. A metal complex having a ligand wherein the number of the aromatic homocyclic ring(s) or aromatic heterocyclic ring(s) (each) obtained by linking the two substituents to each other is made larger tends to have a higher heat resistance.

The metal complex is preferably a metal complex having a ligand in a form that R^{2a} and R^{2b} as well as R^{2c} and R^{2d}, out of the two-substituent pairs, are linked to each other to form an aromatic ring. The metal complex may be a metal complex represented by the formula (A-2).

When any one of R^{4a} to R^{4f}, R^{5a} to R^{5h}, and R^{6a} to R^{6d} in the formula (A-2) is a substituent, examples of the substituent can be the same as exemplified as the substituent about the formula (A-1).

Two substituents of each of pairs of R^{4a} and R^{4b}, R^{4a} and R^{4c}, R^{4d} and R^{4e}, R^{4d} and R^{4f}, R^{5a} and R^{5b}, R^{5b} and R^{5c}, R^{5c} and R^{5d}, R^{5e} and R^{5f}, R^{5f} and R^{5g}, R^{5g} and R^{5h}, R^{4b} and R^{6a}, R^{4e} and R^{6b}, R^{4c} and R^{6c}, and R^{4f} and R^{6d} may be linked to each other to form a ring. Examples of the ring include hydrocarbon rings such as cyclohexene ring, benzene ring, naphthalene ring, and anthracene ring and acenaphthene ring; and aromatic heterocyclic rings such as pyran ring, furan ring, pyridine ring, pyrazine ring, pyrazolyl ring, imidazolyl ring, oxazole ring, isooxazole ring, thiazole ring, isothiazole ring and thiophene ring. Of these rings, monocyclic aromatic homocyclic rings or monocyclic aromatic heterocyclic rings are preferred. In a case where the two substituents of any one of the pairs in the formula (A-2) are combined with each other to form a monocyclic aromatic homocyclic ring or monocyclic aromatic heterocyclic ring, the metal complex corresponds to a complex wherein a ring obtained by linking the two substituents of any one of the pairs shown about the formula (A-1) to each other is a condensed polycycle.

Specific examples of the metal complex represented by the formula (A-2) are (a-III) to (a-XI) out of the exemplified formulae (a-I) to (a-XI).

A metal complex wherein R^{4b}, R^{4c}, R^{4e}, R^{4f}, R^{5a}, R^{5d}, R^{5e} and R^{5h} are each a hydrogen atom, that is, a metal complex represented by the formula (A-3), out of metal complexes represented by the formula (A-2), is preferred since the metal complex of the invention can be obtained at low costs for the following advantage: about each of a phenol compound and a diamine compound for deriving the ligand of the metal complex, a material that is industrially available with ease can be used.

The following describes the metal complex represented by the formula (B-1), which is a second embodiment of the invention. The metal complex is a product wherein M³(s), which is/are (each) a transition metal element or typical metal element, form(s) a complex by aid of a ligand having four heteroatoms and two oxygen atoms. The bond through which any one of the oxygen atoms and (any one of) the metal atom(s) are linked to each other is a coordinate bond or ion bond. When two metal atoms are present therein, bridging coordination may be attained between the two metals. The coordination state of the two metal atoms M³s and the ligand in this metal complex is schematically illustrated in the following formula (I) about a case where in the formula (B-1), R¹ to R¹⁰ are each a hydrogen atom, Y¹ and Y² are each —N═, P¹ is combined with Y¹ and carbon atoms adjacent thereto so as to form a pyridine ring, P² is combined with Y² and carbon atoms adjacent thereto so as to form a pyridine ring, and P¹ and P² are linked to each other so as to form a benzene ring also:

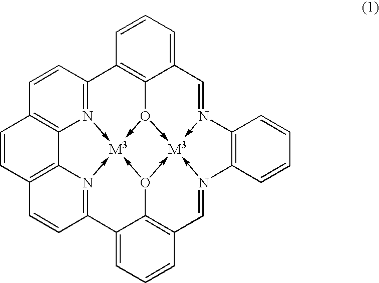

When M³(s) is/are (each) a transition metal atom, the metal atom is preferably titanium, vanadium, chromium, manganese, iron, cobalt, nickel, copper, zinc, zirconium, niobium, molybdenum, technetium, ruthenium, rhodium, palladium, silver, tantalum, tungsten, rhenium, osmium, iridium, platinum or gold, more preferably titanium, vanadium, chromium, manganese, iron, cobalt, nickel, copper, zinc, zirconium, niobium, molybdenum, ruthenium, rhodium, palladium or silver, in particular preferably titanium, vanadium, chromium, manganese, iron, cobalt, nickel, copper or zinc. These transition metal atoms may be in a non-charged state or in a charged ion state.

When M³(s) is/are (each) a typical metal atom, specific examples thereof include aluminum, gallium, germanium, indium, tin, antimony, thallium, lead, and bismuth. These typical metal atoms may also be in a non-charged state or in a charged ion state.

M³(s) described in the formula (B-1) is/are (each) preferably a metal atom selected from the above-mentioned transition metal atoms. When m is 2, two M³s may be the same as or different from each other.

The following describes the ligand of the metal complex represented by the formula (B-1). In the formula (B-1), R¹ to R¹⁰ are each independently a hydrogen atom or substituent. When any one of R¹ to R¹⁰ is a substituent, examples of the substituent can be the same groups as exemplified as the substituent about the formula (A-1).

Two substituents of each of pairs of R¹ and R², R² and R³, R⁴ and R⁵, R⁵ and R⁶, R³ and R⁷, R⁶ and R¹, and R⁸ and R⁹ may be linked to each other to form a ring.

Examples of the ring include hydrocarbon rings such as cyclohexane ring, benzene ring, naphthalene ring, anthracene ring, and acenaphthene ring; and aromatic heterocyclic rings such as furan ring and thiophene ring.

The ring formed by linking the two substituents of any one of the pairs to each other may further have a substituent. Examples of the substituent can be the same groups as exemplified as the substituent about the formula (A-1).

Y¹ and Y² each independently represent

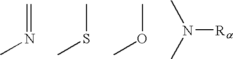

wherein R_{α} is a hydrogen atom or a hydrocarbon group having 1 to 4 carbon atoms. P¹ is a group of atoms necessary for being combined with Y¹ and carbon atoms adjacent thereto so as to form an aromatic heterocyclic ring, P² is a group of atoms necessary for being combined with Y² and carbon atoms adjacent thereto so as to form an aromatic heterocyclic ring, and P¹ and P² may be linked to each other to form an additional ring. Specific examples of the aromatic heterocyclic ring include pyridine, pyrazine, pyrimidine, pyrrole, furan, thiophene, thiazole, imidazole, oxazole, triazole, isoindole, benzofuran, benzothiophene, isoquinoline, and quinazoline. The aromatic heterocyclic ring is preferably pyridine, pyrazine, pyrimidine, pyrrole, furan or thiophene, more preferably pyridine, pyrrole, furan, thiophene.

When P¹ and P² are linked to each other to form an additional ring, the metal complex preferably has a structure selected from the following structures (2-a) to (2-i), and more preferably has a structure from the structures (2-a) to (2-d):

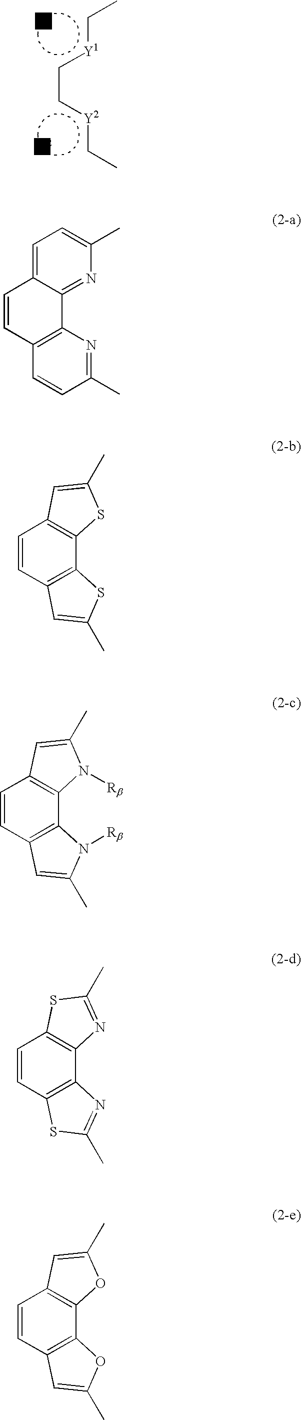

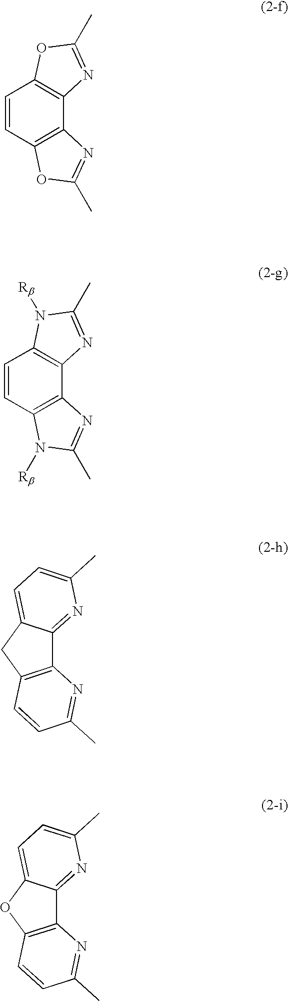

Wherein, R_{β}s each represent a hydrogen atom or a hydrocarbon group having 1 to 30 carbon atoms.

Each of the above-mentioned ring structures made from P¹ and P² may have a substituent. Examples of the substituent can be the same as given as the specific examples of the above-mentioned substituent.

Examples of the metal complex represented by the formula (B-1) include metal complexes having ligand skeleton structures (B-I) to (B-IX) illustrated below. Their transition metal atoms are not illustrated, and their electric charges are omitted. In the examples illustrated below, Me, Et and t-Bu represent methyl, ethyl and tert-butyl groups, respectively.

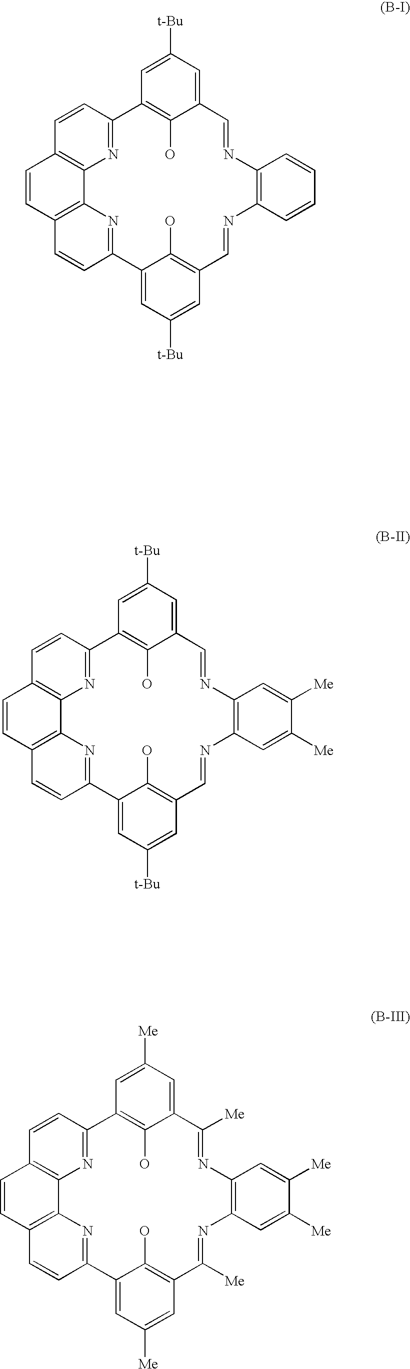

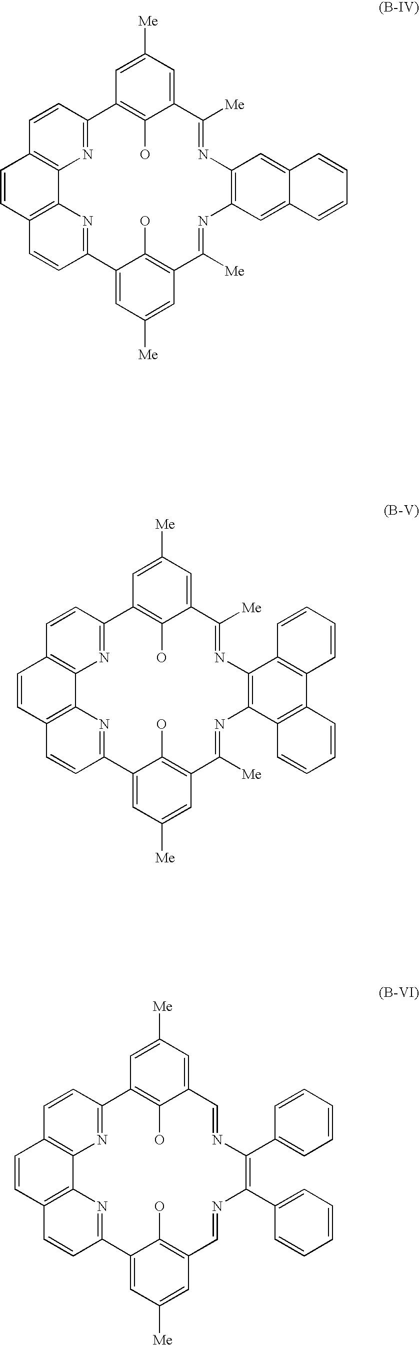

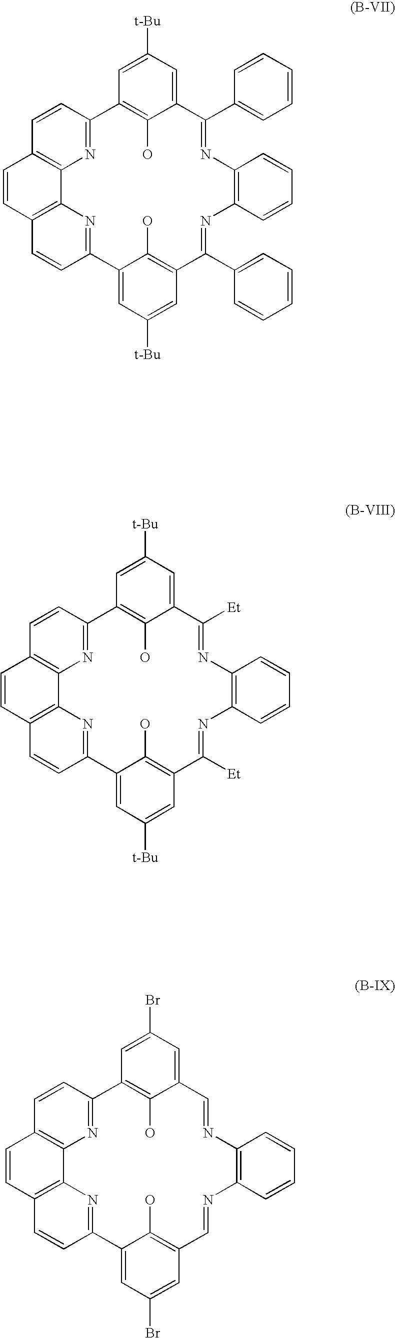

Of metal complexes each represented by the formula (B-1), which are each according to the second embodiment, a metal complex represented by the formula (B-2) is preferred.

When any one of R¹¹ to R²⁶ in the formula (B-1) is a substituent, examples of the substituent can be the same as exemplified as the substituent about the formula (A-1).

When two substituents of each of pairs of R¹¹ and R¹⁴, R¹¹ and R¹², R¹² and R¹³, R¹³ and R¹⁷, R¹⁴ and R¹⁵, R¹⁵ and R¹⁶, R¹⁶ and R²⁰, R¹⁷ and R¹⁸, R¹⁸ and R¹⁹, R²⁰ and R²¹, R²¹ and R²², and R²⁴ and R²⁵ are linked to each other to form a ring, the ring may further have a substituent.

In the formulae (A-1), (A-2), (A-3), (B-1) and (B-2), X(s) is/are (each) a neutral molecule, or a counter ion which makes the metal complex electrically neutral. The neutral molecule is a molecule capable of undergoing solvation to form a solvation salt, or a ligand other than the cyclic ligands in the formulae (A-1), (A-2), (A-3), (B-1) and (B-2). Specific examples of the neutral molecule include water, methanol, ethanol, n-propanol, isopropyl alcohol, 2-methoxyethanol, 1,1-dimethylethanol, ethylene glycol, N,N′-dimethylformamide, N,N′-dimethylacetoamide, N-methyl-2-pyrrolidone, dimethylsulfoxide, acetone, chloroform, acetonitrile, benzonitrile, triethylamine, pyridine, pyrazine, diazabicyclo[2,2,2]octane, 4,4′-bipyridine, tetrahydrofuran, diethyl ether, dimethoxyethane, methyl ethyl ether, and 1,4-dioxane. The molecule is preferably water, methanol, ethanol, isopropyl alcohol, ethylene glycol, N,N′-dimethylformamide, N,N′-dimethylacetoamide, N-methyl-2-pyrrolidone, chloroform, acetonitrile, benzonitrile, triethylamine, pyridine, pyrazine, diazabicyclo[2,2,2]octane, 4,4′-bipyridine, tetrahydrofuran, dimethoxyethane, or 1,4-dioxane.

Usually, the transition metals M¹, M² and M³(s) each have a positive charge; thus, when X(s) is/are (each) an ion, an anion which makes this charge electrically neutral is selected. Examples thereof include the following ions: fluorine, chlorine, bromine, iodine, sulfide, oxide, hydroxide, hydride, sulfurous acid, phosphoric acid, cyanide, acetic acid, carbonic acid, sulfuric acid, nitric acid, hydrogen carbonic acid, trifluoroacetic acid, thiocyanide, trifluoromethanesulfonic acid, acetyl acetonate, tetrafluoroboric acid, hexafluorophosphoric acid, and tetraphenylboric acid ions. The anion is preferably a chloride, bromide, iodide, oxide, hydroxide, hydrate, phosphoric acid, cyanide, acetic acid, carbonic acid, sulfuric acid, nitric acid, acetyl acetonate, or tetraphenylboric acid ion.

When a plural of Xs are present, they may be the same as or different from each other. They may be in the form that a neutral molecule and an ion coexist.

A process for producing the metal complex represented by the formula (A-1) is described herein.

The metal complex represented by the formula (A-1) can be yielded by condensation of a phenol compound or phenol compounds represented by the following formula (3-a) and/or the following formula (3-b) (hereinafter referred to as the “phenol compound(s)”), which has/have two carbonyl groups at the 2-position and the 6-position thereof and will form a ligand, and a diamine derivative and diamine derivatives represented by the following formula (3-c) and/or the following formula (3-d) (hereinafter referred to as the “diamine compound(s)”) in the presence of a reagent for supplying transition metal atoms (hereinafter referred to as the “metal supplier”):

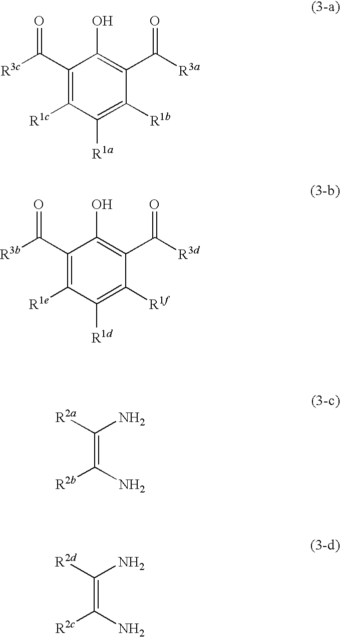

[wherein R^{1a} to R^{1f}, R^{2a} to R^{2d} and R^{3a} to R^{3d} have the same meanings as described above.]

The metal supplier is a compound having the transition metal atoms M¹ and M². Usually, a salt having these transition metals as cations is used.

The following describes a process for producing the metal complex represented by the formula (B-1).

The metal complex represented by the formula (B-1) can be yielded by condensation of a carbonyl compound represented by the following formula (4-a) (hereinafter referred to as the “carbonyl compound”), which has two carbonyl groups and will form a ligand, and a diamine derivative represented by the following formula (4-a) (hereinafter referred to as the “diamine compound”) in the presence of a reagent for supplying a transition metal atom (hereinafter referred to as the “metal supplier”):

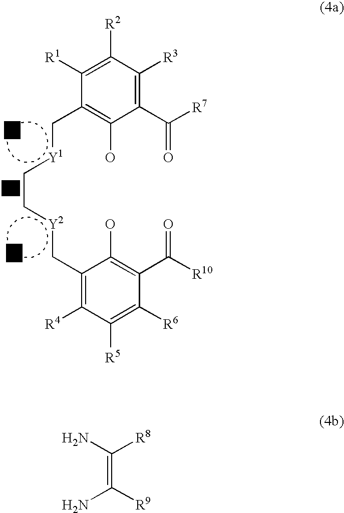

[wherein R¹ to R¹⁰, Y¹, Y², P¹ and P² have the same meanings as described above.]

The metal supplier is a compound having the metal atom M³. Usually, a salt having this transition metal as a cation is used.

As described above, the metal complexes of the invention can each be yielded by condensation of the phenol compound(s) or the carbonyl compound, the diamine compound(s) and the metal supplier in the presence of an appropriate reaction solvent. Specific examples of the reaction solvent include water, methanol, ethanol, 1-propanol, 2-propanol, 1-butanol, ethylene glycol, 2-methoxyethanol, tetrahydrofuran, diethyl ether, 1,2-dimethoxyethane, acetonitrile, benzonitrile, acetone, 1-methyl-2-pyrrolidinone, dimethylformamide, dimethylacetoamide, dimethylsulfoxide, acetic acid, benzene, toluene, xylene, dichloromethane, chloroform, and carbon tetrachloride. A reaction solvent obtained by mixing two or more of these solvents with each other may be used. Preferred is a solvent in which the used phenol compounds) or carbonyl compound, the diamine compound(s) and the metal supplier can be dissolved. The reaction temperature is usually from −10 to 200° C., preferably from 0 to 150° C., in particular preferably from 0 to 100° C. The reaction time is usually from 1 minute to 1 weeks preferably from 5 minutes to 24 hours, in particular preferably from 1 to 6 hours. The reaction temperature and the reaction time may be appropriately optimized in accordance with the kinds of the used phenol compound(s) or carbonyl compound, the diamine compound(s) and the metal supplier.

The metal complexes of the invention may each be produced by use of a method of condensation of the phenol compound(s) or carbonyl compound and the diamine compound(s) or diamine compound in the coexistence of an acid such as hydrochloric acid in a reaction solvent as described above, and then adding thereto a metal salt, as described in a document, Journal of Organic Chemistry, 1999, 64, 1442. The metal salt may be an acetate, a hydrochloride, a sulfate, a carbonate or the like.

The manner for isolating and purifying the produced metal complex from the reaction solution after the reaction may be an optimal manner selected appropriately from known recrystallization, reprecipitation and chromatographic methods. These manners may be combined with each other.

In accordance with the kind of the reaction solvent, the produced metal complex may be precipitated. By separating the precipitated metal complex by filtration or the like and optionally washing and/or drying the separated product, the metal complex can also be isolated and purified.

The metal complexes of the invention may each be used as a polymer having a moiety obtained by removing, from the metal complex, its one or more hydrogen atoms or substituents. For example, the moiety may be bonded, as a side chain, to a polymer which constitutes a main chain. The main-chain-constituting polymer is not particularly limited, and is, for example, an electroconductive polymer, a dendrimer, or a natural polymer. Of these, an electroconductive is particularly preferred. The electroconductive polymer is a generic name of polymeric materials exhibiting metallic or semiconductive electroconductivity (Iwanami Scientific and Chemical Dictionary, 5^{th} version, published in 1998). Examples of the electroconductive polymer include polyacetylene and derivatives thereof, poly-p-phenylene and derivatives, poly-p-phenylenevinylene and derivatives thereof, polyaniline and derivatives, polythiophene and derivatives thereof, polypyrrole and derivatives thereof, polyfluorene and derivatives thereof, polycarbazole and derivatives, polyindole and derivatives, and copolymers of these electroconductive polymers, as described in “Electroconductive Polymers” (written by Shinichi Yoshimura, Kyoritsu Shuppan Co., Ltd.) and “Newest Applied Technology of Electroconductive Polymers” (supervised by Masao Kobayashi, CMC Publishing Co., Ltd.).

The metal complexes of the invention may each be used as a polymer containing, as a recurring unit, a moiety obtained by removing, from the metal complex, its hydrogen atoms and/or substituents.

The metal complexes of the invention each have a high heat resistance and a high acid resistance, and their complex structure is stably maintained even at high temperature and in the presence of a strong acid. Thus, a fall in their catalyst potency is expected to be small.

In particular, the metal complexes are preferable for redox catalysts and others. Specific examples of articles to which they are applied include catalysts for decomposing hydrogen peroxide, catalysts for oxidizing and polymerizing aromatic compounds, catalysts for cleaning exhaust gas or discharged water, redox catalyst layers in dye-sensitization solar cells, catalysts for reducing carbon dioxide, catalysts for producing reformed hydrogen, and oxygen sensors. It appears that the metal complexes can each be used also as an organic EL luminescent material, or an organic semiconductor material of an organic transistor, a dye-sensitization solar cell or the like by use of a matter that the conjugation skeleton thereof is being extended.

The invention will be specifically described by way of the following examples; however, the invention is not limited to these examples.

### Example 1

### Synthesis of Metal Complex (A)

The metal complex (A) was synthesized in accordance with the following reaction formula:

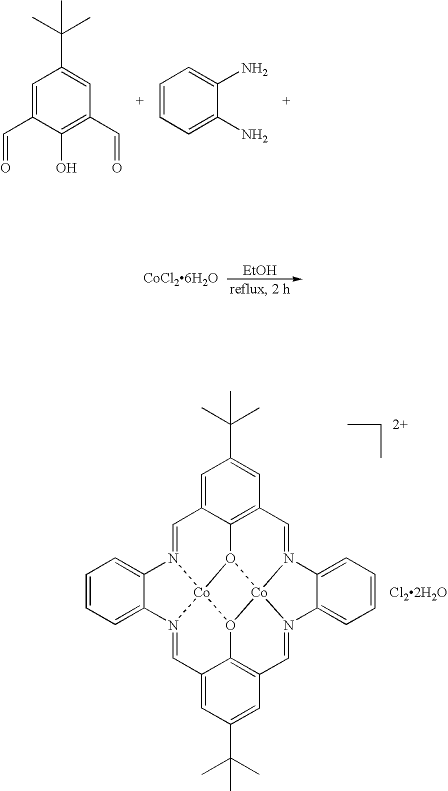

Into a 50-mL eggplant flask was put 10 mL of a solution containing 0.476 g of cobalt chloride hexahydrate and 0.412 g of 4-tert-butyl-2,6-diformylphenol in ethanol in the atmosphere of nitrogen, and then the solution was stirred at room temperature. To this solution was gradually added 5 mL of a solution containing 0.216 g of o-phenylenediamine in ethanol. The mixture was refluxed for 2 hours to produce a brown precipitation. This precipitation was collected by filtration, and dried to yield a metal complex (A) (yielded amount: 0.465 g, yield: 63%). The infrared ray (IR) absorption spectrum of the resultant metal complex (A) is shown in FIG. 1, Elementary analysis values (%): Calcd. for C₃₆H₃₈Cl₂Co₂N₄O₄: C, 55.47; H, 4.91; N, 7.19. Found: C, 56.34; H, 4.83; N, 7.23. In the above-mentioned reaction formula, the expression “Cl₂” denotes that two equivalents of chloride ions are present as counter ions, and the expression “2H₂O” denotes that two equivalents of water molecules are contained as a component which constitutes the metal complex (A).

### Example 2

### Synthesis of Metal Complex (B)

The metal complex (B) was synthesized in accordance with the following reaction formula:

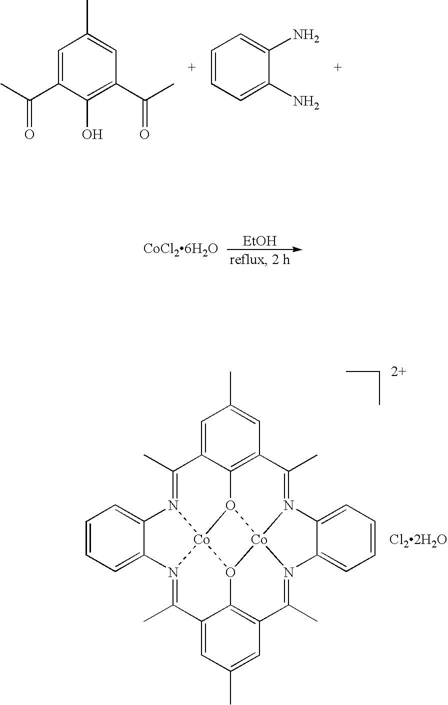

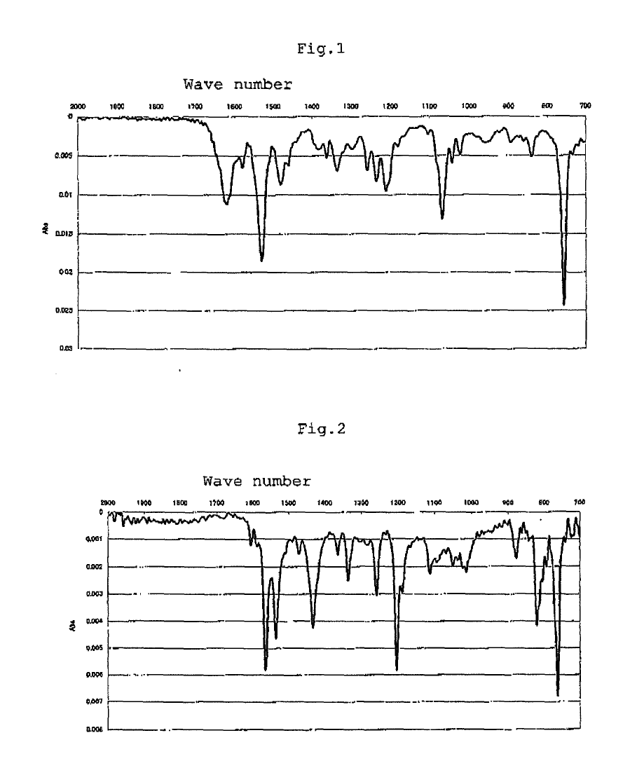

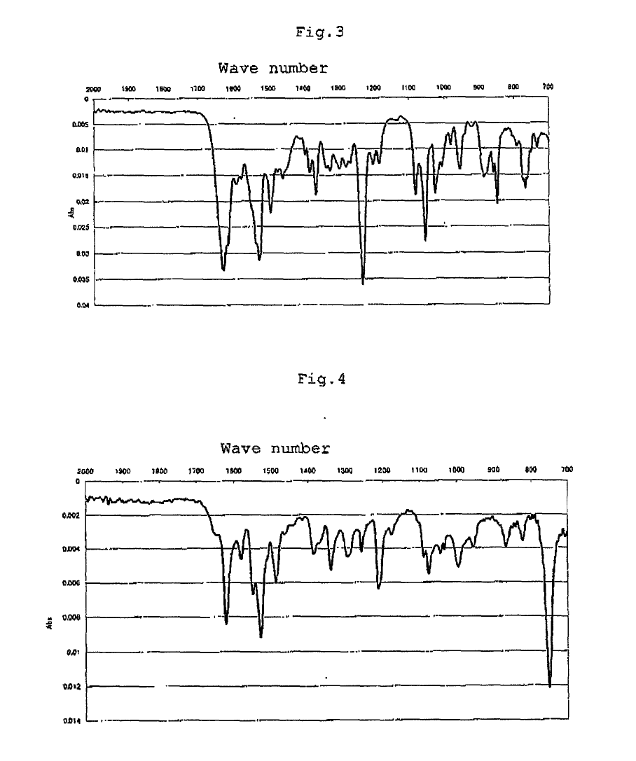

Into a 50-mL eggplant flask was put 5 mL of a solution containing 0.238 g of cobalt chloride hexahydrate and 0.192 g of 4-methyl-2,6-diacetylphenol in ethanol in the atmosphere of nitrogen, and then the solution was stirred at room temperature. To this solution was gradually added 10 mL of a solution containing 0.108 g of o-phenylenediamine in ethanol. The mixture was refluxed for 3 hours to produce a brown precipitation. This precipitation was collected by filtration, and dried to yield a metal complex (B) (yielded amount; 0.129 g, yield; 36%). The infrared ray (IR) absorption spectrum of the resultant metal complex (B) is shown in FIG. 2. Elementary analysis values (%) Calcd. for C₃₄H₃₄Cl₂Co₂N₄O₄: C, 54.34; H, 4.56; N, 7.46. Found: C, 53.57; H, 4.49; N, 7.00. In the above-mentioned reaction formula, the expression “Cl₂” denotes that two equivalents of chloride ions are present as counter ions, and the expression “2H₂O” denotes that two equivalents of water molecules are contained as a component which constitutes the metal complex (B).

### Example 3

### Synthesis of Metal Complex (C)

The metal complex (C) was synthesized in accordance with the following reaction formula:

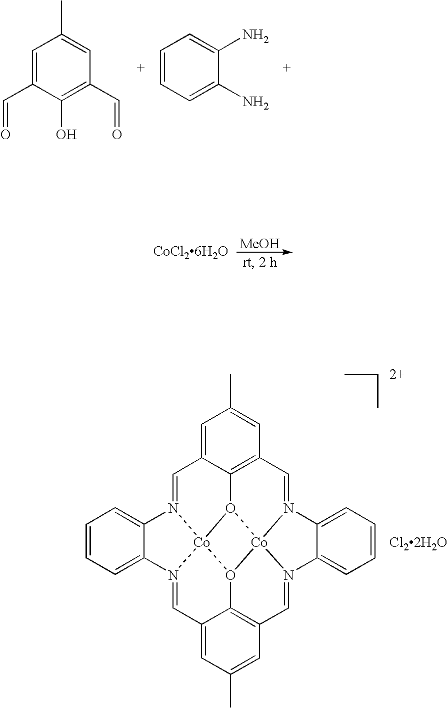

Into a 100-mL eggplant flask was put 25 mL of a solution containing 0.476 g of cobalt chloride hexahydrate and 0.328 g of 4-methyl-2,6-diformylphenol in ethanol in the atmosphere of nitrogen, and then the solution was stirred at room temperature. To this solution was gradually added 5 mL of a solution containing 0.216 g of o-phenylenediamine in ethanol. The mixture was refluxed for 2 hours to produce a brown precipitation. This precipitation was collected by filtration, and dried to yield a metal complex (C) (yielded amount: 0.368 g, yield: 56%). The infrared ray (IR) absorption spectrum of the resultant metal complex (C) is shown in FIG. 3. Elementary analysis values (%): Calcd. for C₃₀H₂₆Cl₂Co₂N₄O₄: C, 51.82; H, 3.77; N, 8.06. Found: C, 52.41; H, 3.95; N, 8.20. In the above-mentioned reaction formula, the expression “Cl₂” denotes that two equivalents of chloride ions are present as counter ions, and the expression “2H₂O” denotes that two equivalents of water molecules are contained as a component which constitutes the metal complex (C).

### Example 4

### Synthesis of Metal Complex (D)

The metal complex (D) was synthesized in accordance with the following reaction formula:

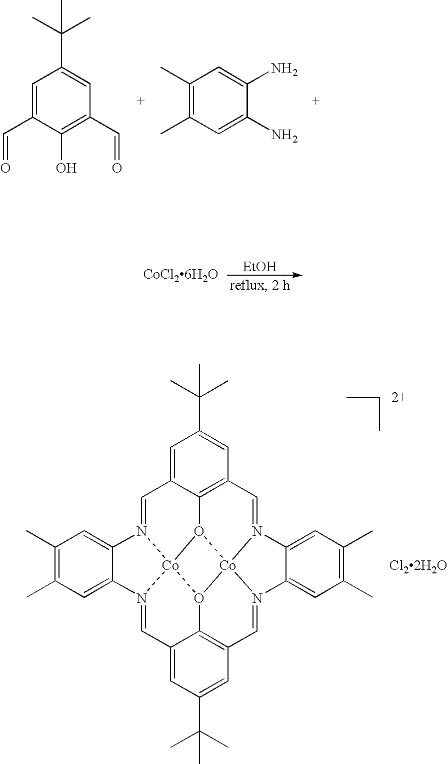

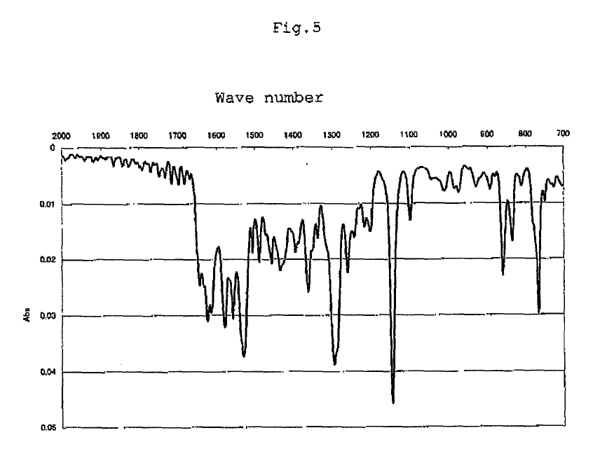

Into a 50-mL eggplant flask was put 10 mL of a solution containing 0.476 g of cobalt chloride hexahydrate and 0.412 g of 4-tert-butyl-2,6-diformylphenol in ethanol in the atmosphere of nitrogen, and then the solution was stirred at room temperature. To this solution was gradually added 10 mL of a solution containing 0.272 g of 4,5-dimethyl-1,2-phenylenediamine in ethanol. The mixture was refluxed for 2 hours to produce a brown precipitation. This precipitation was collected by filtration, and dried to yield a metal complex (D) (yielded amount: 0.513 g, yield: 64%). The infrared ray (IR) absorption spectrum of the resultant metal complex (D) is shown in FIG. 4. Elementary analysis values (%): Calcd. for C₃₆H₃₄Cl₂Co₂N₄O₄: C, 57.50; H, 5.55; N, 6.71. Found; C, 53.85; H, 5.86; N, 5.78. In the above-mentioned reaction formula, the expression “Cl₂” denotes that two equivalents of chloride ions are present as counter ions, and the expression “2H₂O” denotes that two equivalents of water molecules are contained as a component which constitutes the metal complex (D).

### Example 5

### Synthesis of Metal Complex (E)

The metal complex (E) was synthesized in accordance with a reaction formula illustrated below. The following aldehyde, which is a raw material of the complex, was synthesized on the basis of Tetrahedron., 1999, 55, 8377:

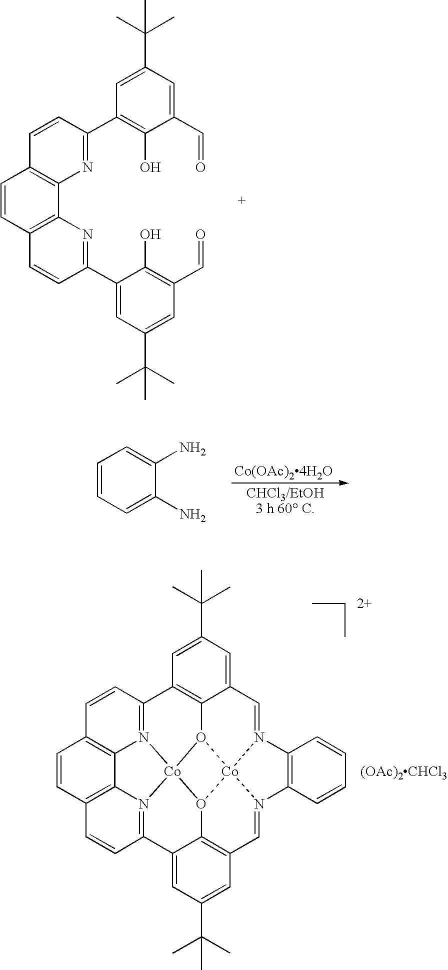

Into a 50-mL eggplant flask was put a mixed solution containing 0.199 g of cobalt acetate tetrahydrate and 0.213 g of the aldehyde compound in 5 mL of chloroform and 5 mL of ethanol in the atmosphere of nitrogen, and then the solution was stirred at 60° C. To this solution was gradually added 5 mL of a solution containing 0.043 g of o-phenylenediamine in ethanol. The mixture was refluxed for 3 hours to produce a brown precipitation. This precipitation was collected by filtration, and dried to yield a metal complex (E) (yielded amount: 0.109 g, yield: 28%).

Elementary analysis values (%): Calcd. for C₄₅H₄₁Cl₃Co₂N₄O₆: C, 56.41; H, 4.31; N, 5.85. Found: C, 58.28; H, 4.81; N, 5.85. ESI-MS[M-CH₃COO]⁺: 779.0.

### Example 6

### Synthesis of Metal Complex (F)

The metal complex (F) was synthesized in accordance with the following reaction formula:

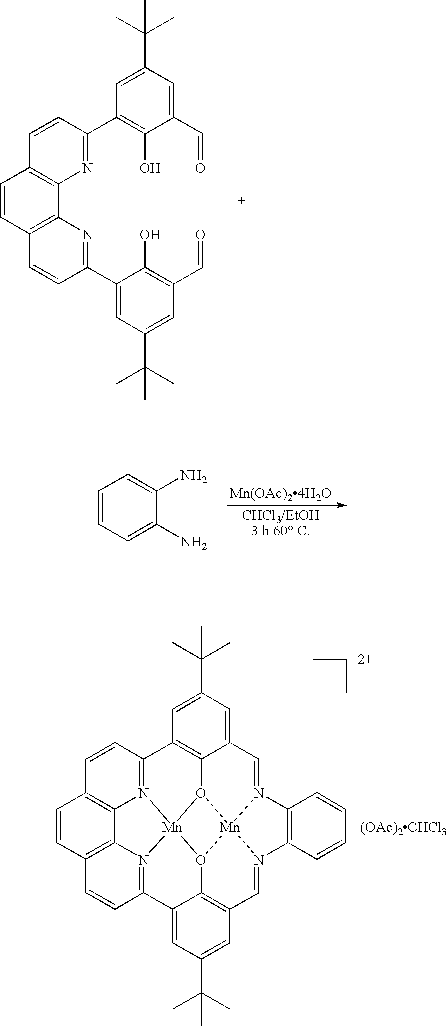

Into a 50-mL eggplant flask was put a mixed solution containing 0.221 g of manganese acetate tetrahydrate and 0.213 g of the aldehyde compound in 10 mL of chloroform and 5 mL of ethanol in the atmosphere of nitrogen, and then the solution was stirred at 60° C. To this solution was gradually added 5 mL of a solution containing 0.043 g of o-phenylenediamine in ethanol. The mixture was refluxed for 3 hours to produce a brown precipitation. This precipitation was collected by filtration, and dried to yield a metal complex (F) (yielded amount: 0.166 g, yield; 44%). ESI-MS[M-CH₃COO]⁺: 771.0.

### Synthesis Example 1

### Synthesis of Metal Complex (G)

A metal complex (G), was synthesized in a method described in Australian Journal of Chemistry, 1970, 23, 2225, as in the following reaction formula:

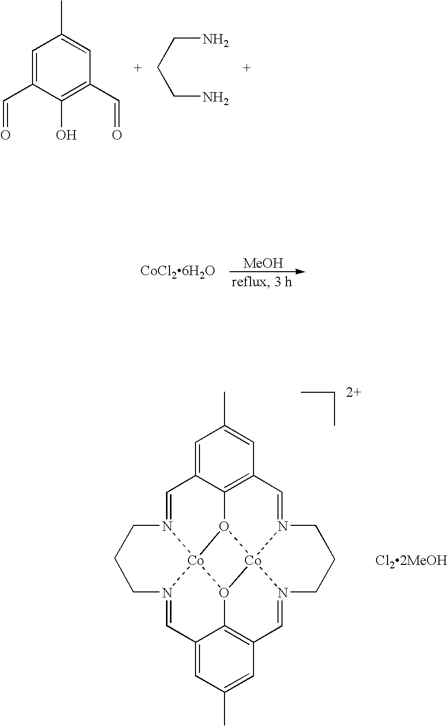

Into a 100-mL eggplant flask was put 50 mL of a solution containing 1.9 g of cobalt chloride hexahydrate and 1.31 g of 4-methyl-2,6-diformylphenol in methanol in the atmosphere of nitrogen, and then the solution was stirred at room temperature. To this solution was gradually added 20 mL of a solution containing 0.59 g of 1,3-propanediamine in methanol. The mixture was refluxed for 3 hours to produce a brown precipitation. This precipitation was collected by filtration, and dried to yield a metal complex (E) (yielded amount: 1.75 g, yield: 74%).

Elementary analysis values (%): Calcd. for C₂₆H₃₄Cl₂Co₂N₄O₄; C, 47.65; H, 5.23; N, 8.55. Found: C, 46.64; H, 5.02; N, 8.58. In the above-mentioned reaction formula, the expression “Cl₂” denotes that two equivalents of chloride ions are present as counter ions, and the expression “2MeOH” denotes that two equivalents of methanol molecules are contained as a component which constitutes the metal complex (G).

### Synthesis Example 2

### Synthesis of Metal Complex (H)

A metal complex (H) was synthesized in accordance with the following reaction formula (H):

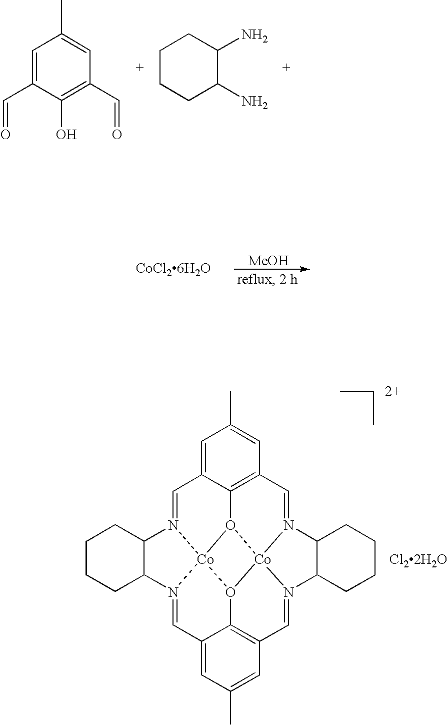

Into a 50-mL eggplant flask was put 10 mL of a solution containing 0.476 g of cobalt chloride hexahydrate and 0.328 g of 4-methyl-2,6-diformylphenol in methanol in the atmosphere of nitrogen, and then the solution was stirred at room temperature. To this solution was gradually added 5 mL of a solution containing 0.228 g of trans-1,2-cyclohexanediamine in methanol. The mixture was refluxed for 2 hours to produce a brown precipitation. This precipitation was collected by filtration, and dried to yield a metal complex (F) (yielded amount: 0.141 g, yield: 21%).

Elementary analysis values (%): Calcd. for C₃₀H₃₈Cl₂Co₂N₄O₄: C, 50.93; H, 5.41; N, 7.92. Found: C, 49.60; H, 5.47; N, 8.04. In the above-mentioned reaction formula, the expression “Cl₂” denotes that two equivalents of chloride ions are present as counter ions, and the expression “2H₂O” denotes that two equivalents of water are contained as a component which constitutes the metal complex (H).

### [Acid Resistance Test of Metal Complex (A) at Room Temperature]

About the metal complex (A), sulfuric acid was used at room temperature (25° C.) to make an acid resistance test. The metal complex (A) was weighed out in an amount of 2.84 mg, and the weighed complex was dissolved into 20 mL of methanol. The solution was measured out in a volume of 9.0 mL, and the measured solution was added to 1.0 mL of a 1-M aqueous solution of sulfuric acid. The solution was rapidly stirred, and then a volume of 0.3 mL was collected. The collected solution was diluted 10 times, and the diluted solution was charged into a cell. An ultraviolet-visible spectrophotometer (V-530, manufactured by JASCO Corporation) was used to observe a change in the ultraviolet-visible absorption thereof at room temperature with the passage of time. Values of the absorbance at a wavelength of 454 nm are shown in Table 1. From this result, the following was made clear: about the metal complex (A), the absorbance was hardly changed even in the presence of the acid; thus, the complex structure thereof was maintained.

TABLE 1

| | Absorbance at 454 nm |
|---|---|
| Just after the charging | 0.472 |
| After 30 minutes | 0.471 |
| After 1 hour | 0.471 |
| After 2 hours | 0.470 |

About the metal complex (G), in the same way as about the metal complex (A), sulfuric acid was used at room temperature to make an acid resistance test, and then a change in the UV absorption was observed with the passage of time. Values of the absorbance at a wavelength of 371 nm are shown in Table 2. About the metal complex (G), the absorbance was decreased in the presence of the acid with the passage of time. From this matter, it was made clear that the complex structure was changed.

TABLE 2

| | Absorbance at 371 nm |
|---|---|
| Just after the charging | 0.255 |
| After 30 minutes | 0.110 |
| After 1 hour | 0.077 |
| After 2 hours | 0.055 |

About the metal complex (E), sulfuric acid was used at 60° C. to make an acid resistance test. The metal complex (E) was weighed out in an amount of 7.90 mg, and the weighed complex was dissolved into 36 ml of methanol. The solution was measured out in a volume of 9.0 mL, and the measured solution was added to 1.0 mL of a 1-M aqueous solution of sulfuric acid. The solution was rapidly stirred, and then a volume of 0.3 mL was collected. The collected solution was diluted 10 times, and the diluted solution was charged into a cell. A lid thereof was closed, and the cell was heated to 60° C. A spectrophotometer (Cary 5E, manufactured by Varian Technologies Japan Limited) was used to observe a change in the ultraviolet and visible ray absorption of the solution with the passage of time. Values of the absorbance at a wavelength of 445 nm, and the absorbance ratios from the time just after the charging to subsequent times are shown in Table 3.

TABLE 3

| | Absorbance at 445 nm | Absorbance ratio |
|---|---|---|
| Just after the charging | 0.464 | 1.00 |
| After 2 hours | 0.332 | 0.72 |
| After 3 hours | 0.268 | 0.58 |
| After 4 hours | 0.227 | 0.49 |

About the metal complex (A), in the same way as about the metal complex (E), sulfuric acid was used at 60° C. to make an acid resistance test, and then a change in the UV absorption was observed with the passage of time. Values of the absorbance at a wavelength of 455 nm, and the absorbance ratios from the time just after the charging to subsequent times are shown in Table 4.

TABLE 4

| | Absorbance at 455 nm | Absorbance ratio |
|---|---|---|
| Just after the charging | 0.363 | 1.00 |
| After 2 hours | 0.265 | 0.72 |
| After 3 hours | 0.136 | 0.37 |
| After 4 hours | 0.098 | 0.27 |

From Tables 3 and 4, it is demonstrated that about the metal complex (E), a decrease in the absorbance in the presence of the acid with the passage of time is restrained, so that the degree that the metal complex is decomposed is smaller, as compared with that about the metal complex (A).

### [Heat Resistance Test of Metal Complex (C)]

About the metal complex (C), a thermogravimetric/differential-thermal analyzer (EXSTAR-6300, manufactured by Seiko Instruments Inc.) was used to measure a change in the weight (TGA) when the complex was thermally treated. The weight reduction ratio at 800° C. was measured from the ratio relative to the initial weight supplied to the measurement. The measurement was made in the atmosphere of nitrogen at 40 to 800° C. (temperature-raising rate: 10° C./min.), and an aluminum dish was used for the thermal treatment. The weight reduction ratio is shown in Table 5.

### [Heat Resistance Test of Metal Complex (E)]

About the metal complex (E), in the same way as about the metal complex (C), the weight change (TGA) was measured when the complex was thermally treated. The weight reduction ratio of the metal complex (E) at 800° C. is shown in Table 5.

### [Heat Resistance Test of Metal Complex (G)]

About the metal complex (G), in the same way as about the metal complex (C), the weight change (TGA) was measured when the complex was thermally treated. The weight reduction ratio of the metal complex (G) at 800° C. is shown in Table 5,

### [Heat Resistance Test of Metal Complex (H)]

About the metal complex (H), in the same way as about the metal complex (C), the weight change (TGA) was measured when the complex was thermally treated. The weight reduction ratio of the metal complex (H) at 800° C. is shown in Table 5.

TABLE 5

| | Weight reduction ratio at 800° C. |
|---|---|
| Metal complex (C) | 36.72 |
| Metal complex (E) | 46.26 |
| Metal complex (G) | 50.41 |
| Metal complex (H) | 57.12 |

From Table 5, the following was made clear: the metal complexes (C) and (E) each gave a smaller weight reduction ratio and a better heat resistance than the metal complexes (G) and (H).

1. A metal complex represented by the following formula (A-1): 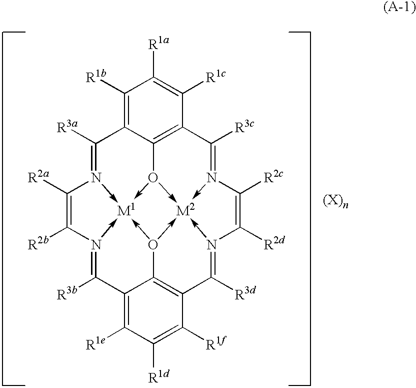
wherein one of M¹ and M² represents a transition metal atom belonging to Groups 6 to 9 of the long-period form periodic table, the other thereof represents a transition metal atom belonging to Groups 6 to 11 of the long-period form periodic table, and M¹ and M² may be the same as or different from each other; R^{1a} to R^{1f}, R^{2a} to R^{2d}, and R^{3a} to R^{3d} each independently represent a hydrogen atom or a substituent, and two substituents of each of pairs of R^{1a} and R^{1b}, R^{1a} and R^{1c}, R^{1d} and R^{1e}, R^{1d} and R^{1f}, R^{2a} and R^{2b}, R^{2c} and R^{2d}, R^{1b} and R^{3a}, R^{1c} and R^{3c}, R^{1e} and R^{3b} and R^{1f} and R^{3d} may be linked to each other to form a ring; X is a counter ion which makes the metal complex electrically neutral, or a neutral molecule; n is the number of X(s) present in the complex and represents an integer of 0 to 4 provided that when n is an integer of 2 to 4, a plural of Xs may be the same as or different from each other; and the symbols “→” each represent a coordinate bond or ion bond to M¹ or M².

2. The metal complex according to claim 1, wherein at least one of M¹ and M² is a transition metal ion belonging to the fourth period of the long-period form periodic table.

3. A metal complex represented by the following formula (A-2): 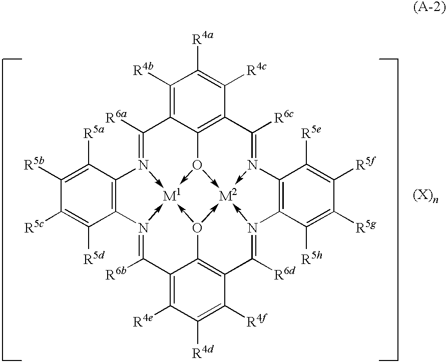
wherein one of M¹ and M² represents a transition metal atom belonging to Groups 6 to 9 of the long-period form periodic table, the other thereof represents a transition metal atom belonging to Groups 6 to 11 of the long-period form periodic table, and M¹ and M² may be the same as or different from each other; R^{4a} to R^{4f}, R^{5a} to R^{5h}, and R^{6a} to R^{6d} each independently represent a hydrogen atom or a substituent, and two substituents of each of pairs of R^{4a} and R^{4b}, R^{4a} and R^{4c}, R^{4d} and R^{4e}, R^{4d} and R^{4f}, R^{5a} and R^{5b}, R^{5b} and R^{5c}, R^{5c} and R^{5d}, R^{5e} and R^{5f}, R^{5f} and R^{5g}, R^{5g} and R^{5h}, R^{4b}, R^{6a}, R^{4e} and R^{6b}, R^{4c} and R^{6c} and R^{4f} and R^{6d} may be linked to each other to form a ring; X is a counter ion which makes the metal complex electrically neutral, or a neutral molecule; n is the number of X(s) present in the complex and represents an integer of 0 to 4 provided that when n is an integer of 2 to 4, a plural of Xs may be the same as or different from each other; and the symbols “→” each represent a coordinate bond or ion bond to M¹ or M².

4. The metal complex according to claim 3, wherein at least one of M¹ and M² is a transition metal ion belonging to the fourth period of the long-period form periodic table.

5. A metal complex represented by the following formula (A-3): 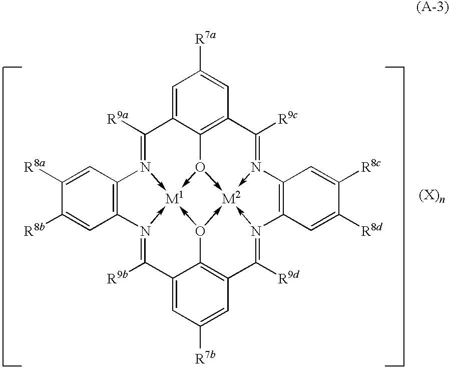
wherein one of M¹ and M² represents a transition metal atom belonging to Groups 6 to 9 of the long-period form periodic table, the other thereof represents a transition metal atom belonging to Groups 6 to 11 of the long-period form periodic table, and M¹ and M² may be the same as or different from each other; R^{7a}, R^{7b}, R^{8a} to R^{8d} and R^{9a} to R^{9d} each independently represent a hydrogen atom or a substituent, and two substituents of each of pairs of R^{8a} and R^{8b}, and R^{8c} and R^{8d} may be linked to each other to form a ring; X is a counter ion which makes the metal complex electrically neutral, or a neutral molecule; n is the number of X(s) present in the complex and represents an integer of 0 to 4 provided that when n is an integer of 2 to 4, a plural of Xs may be the same as or different from each other; and the symbols “→” each represent a coordinate bond or ion bond to M¹ or M².

6. The metal complex according to claim 5, wherein at least one of M¹ and M² is a transition metal ion belonging to the fourth period of the long-period form periodic table.

7. A metal complex represented by the following formula (B-1): 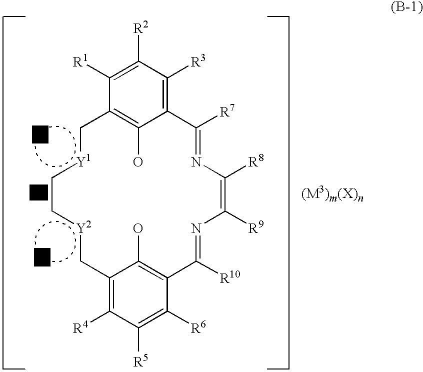
wherein R¹ to R¹⁰ each independently represent a hydrogen atom or a substituent; two substituents of each of pairs of R¹ and R², R² and R³, R⁴ and R⁵, R⁵ and R⁶, R³ and R⁷, R⁶ and R¹⁰, and R⁸ and R⁹ may be linked to each other to form a ring; Y¹ and Y² each independently represent 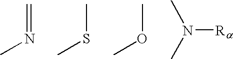
wherein R_{α} is a hydrogen atom or a hydrocarbon group having 1 to 4;
P¹ is a group of atoms necessary for being combined with Y¹ and carbon atoms adjacent thereto so as to form an aromatic heterocyclic ring, P² is a group of atoms necessary for being combined with Y² and carbon atoms adjacent thereto so as to form an aromatic heterocyclic ring, and P¹ and P² may be linked to each other to form an additional ring; M³ represents a transition metal atom or a typical metal atom; m represents 1 or 2 provided that when m is 2, two M³s may be the same as or different from each other; X is a counter ion which makes the metal complex electrically neutral, or a neutral molecule; and n is the number of X(s) present in the complex and represents an integer of 0 or more provided that when n is an integer of 2 or more, a plural of Xs may be the same as or different from each other.

8. The metal complex according to claim 7, wherein M³ is a transition metal atom.

9. A metal complex represented by the following formula (B-2): 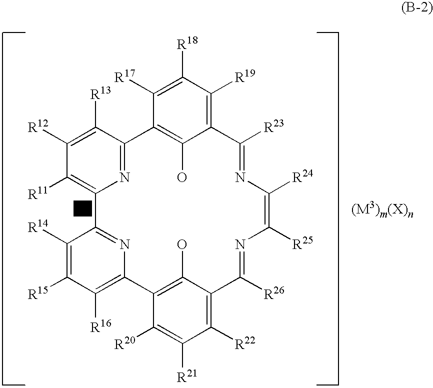
wherein R¹¹ to R²⁶ each independently represent a hydrogen atom or a substituent; two substituents of each of pairs of R¹¹ and R¹⁴, R¹¹ and R¹², R¹² and R¹³, R¹³ and R¹⁷, R¹⁴ and R¹⁵, R¹⁵ and R¹⁶, R¹⁶ and R²⁰, R¹⁷ and R¹⁸, R¹⁸ and R¹⁹, R²⁰ and R²¹, R²¹ and R²², and R²⁴ and R²⁵ may be linked to each other to form a ring; M³ represents a transition metal atom or a typical metal atom; m represents 1 or 2 provided that when m is 2, two M³s may be the same as or different from each other; X is a counter ion which makes the metal complex electrically neutral, or a neutral molecule; and n is the number of X(s) present in the complex and represents an integer of 0 or more provided that when n is an integer of 2 or more, a plural of Xs may be the same as or different from each other.

10. The metal complex according to claim 9, wherein M³ is a transition metal atom.

11. A polymer comprising a moiety obtained by removing, from a metal complex as recited in claim 1, its hydrogen atom or its substituent.

12. A catalyst comprising a metal complex as recited in claim 1.

13. A catalyst comprising a polymer as recited in claim 11.
